(19) United States
(12) Patent Application Publication
(10) Pub. No.: US 2024/0298925 A1
(43) Pub. Date: Sep. 12, 2024

(54) NON-CONTACT OCULAR MICROTREMOR MONITOR AND METHODS

(71) Applicant: Covidien LP, Mansfield, MA (US)

(72) Inventors: Philip C. Smit, Hamilton (GB); Paul S. Addison, Edinburgh (GB); Keith Batchelder, Boulder, CO (US)

(21) Appl. No.: 18/261,996

(22) PCT Filed: Jan. 25, 2022
(86) PCT No.: PCT/US2022/070342

§ 371 Date: Jul. 18, 2023

Related U.S. Application Data
(60) Provisional application No. 63/141,129, filed on Jan. 25, 2021.

(51) Int. Cl. A61B 5/11; A61B 5/00; A61B 90/00

(52) U.S. Cl. CPC A61B 5/1106; A61B 5/0077; A61B 90/39; A61B 2090/3945

(57) ABSTRACT

In some examples, a method including receiving, from an image capture device, a sequence of images of an eye region of a patient; determining, using processing circuitry, a motion of a feature within the eye region based on the received sequence of images; and determining, using the processing circuitry, a depth of anesthesia of the patient based on the determined motion is disclosed.

This application claims priority of U.S. Provisional Patent Application Ser. No. 63/141,129, entitled “NON-CONTACT OCULAR MICROTREMOR MONITOR AND METHODS,” and filed on Jan. 25, 2021, the entire content of which is incorporated herein by reference.

### TECHNICAL FIELD

The disclosure relates, in some examples, to patient monitoring.

### BACKGROUND

A patient undergoing a medical procedure may be anesthetized by receiving one or more pharmacological anesthetic agents. Different anesthetic agents may produce different effects, such as sedation or hypnosis (e.g., the lack of consciousness or awareness of the surrounding world), analgesia (e.g., the blunting or absence of pain), or paralysis (e.g., muscle relaxation, which may or may not result in lack of voluntary movement by the patient). Anesthetic agents may provide one or more of these effects and to varying extents on different patients. For example, neuromuscular blocking agents may provide potent paralysis, but no sedation or analgesia. Opioids may provide analgesia and relatively light levels of sedation. Volatile anesthetic agents may provide relatively significant levels of sedation and much smaller levels of analgesia, while the intravenous sedative agent propofol may provide sedation but essentially no analgesia. For this reason, anesthesia providers may simultaneously administer several of these agents to a patient to provide the desired set of effects. For example, an anesthesia provider may administer to a patient a volatile anesthetic agent for its sedative effect, a neuromuscular blocking agent for paralysis and an opioid agent to provide analgesia. In general, the magnitude of the effects provided by these agents are dose-dependent; the higher the dose, the more profound the effect.

### SUMMARY

The present disclosure is directed to, in some examples, a non-contact method and system for measuring and monitoring ocular microtremors of a patient. The example method and system may monitor ocular microtremors to determine a patient's depth of anesthesia (DOA) (also referred to as depth of consciousness in some examples) before, during, and/or after a medical procedure (e.g., a surgical procedure). For example, the systems and techniques may be used, e.g., by a clinician or other medical personnel, to evaluate a patient before or during a medical procedure (e.g., during which the patient is anesthetized for a period of time while a surgeon operates on the patient) to determine a DOA index score and/or other indicator for the patient, which is indicative of a determined DOA for the patient, e.g., for a particular time or time period.

In some examples described herein, processing circuitry of a medical device system is configured to generate a DOA index score for a patient based ocular microtremors of the patient indicated by signals received by the processing circuitry. In some examples, the DOA index score may be determined based on characteristic frequencies of ocular microtremors (OMTs) or other characteristics of the patient's OMTs that may be determined based on images or a sequence of images including movement of a patient's eye related to OMTs.

In some examples, the disclosure is directed to a method comprising receiving, from an image capture device, a sequence of images of an eye region of a patient; determining, using processing circuitry, a motion of a feature within the eye region based on the received sequence of images; and determining, using the processing circuitry, a depth of anesthesia of the patient based on the determined motion.

In some examples, the disclosure is directed to a system comprising an image capture device; and processing circuitry configured to receive, from an image capture device, a sequence of images of an eye region from the image capture device; determine a motion of a feature within the eye region based on the received sequence of images; and determine a depth of anesthesia based on the determined motion.

In some examples, the disclosure is directed to a method comprising receiving a sequence of images of an eye region; and determining a motion of a feature within the eye region based on the received sequence of images.

In some examples, the disclosure is directed to a system comprising an image capture device; and processing circuitry configured to receive, from an image capture device, a sequence of images of an eye region from the image capture device; and determine a motion of a feature within the eye region based on the received sequence of images.

Thus, the disclosed examples provide a non-contact method and apparatus for measuring and monitoring ocular microtremors.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

For a better understanding of the various described examples, reference should be made to the Detailed Description below, in conjunction with the following drawings in which like reference numerals refer to corresponding parts throughout the figures. The figures are not drawn to scale unless indicated otherwise.

FIG. 1 is an illustration depicting an example ocular microtremor (OMT) monitoring system, in accordance with the techniques described in this disclosure.

FIG. 2 is a flowchart illustrating an example method of determining a depth of anesthesia, in accordance with techniques described in this disclosure.

FIG. 3A is an illustration depicting an example image frame that includes a region of interest (ROI) including a feature, in accordance with techniques described in this disclosure.

FIG. 3B is a zoomed-in example of a ROI including a feature, in accordance with techniques described in this disclosure.

FIG. 3C is an example plot of a ROI sum profile including a feature shift difference value, in accordance with techniques described in this disclosure.

FIGS. 4A-4C are example plots of a ROI sum profile at three different times each including a feature shift difference value that depends on the position of the feature in the ROI, in accordance with the techniques described in this disclosure.

FIG. 5 is an example plot of a signal pertaining to motion of a feature included in a sequence of images, in accordance with the techniques described in this disclosure.

FIG. 6 is an example plot of a filtered signal pertaining to motion of a feature included in a sequence of images, in accordance with the techniques described in this disclosure.

FIG. 7 is an illustration depicting an example image frame that includes a plurality of regions of interest (ROIs) that include features added to the eyelid, in accordance with the techniques described in this disclosure.

FIG. 8 is a flowchart illustrating an example method of determining a depth of anesthesia, in accordance with techniques described in this disclosure.

FIG. 9 is an illustration of an example eye region including eyelid and light source feature 902, in accordance with techniques described in this disclosure.

FIG. 10 is an example plot of a cross-section of the angular intensity profile of a light source that may be used in a system for capturing OMT signals, in accordance with techniques described in this disclosure.

FIG. 11 is an example plot of a cross-section of another angular intensity profile of a light source that may be used in a system for capturing OMT signals from the perspective of an observing detector, in accordance with techniques described in this disclosure.

FIG. 12 is an illustration of a system for capturing OMT signals, in accordance with techniques described in this disclosure.

FIG. 13 is a schematic of an example far-field intensity angular output distribution of a light source that may be used in a system for capturing OMT signals, in accordance with techniques described in this disclosure.

FIG. 14 is a flowchart illustrating an example method of determining a depth of anesthesia, in accordance with techniques described in this disclosure.

FIG. 15 is an illustration of an example eye region including eyelid and patch, in accordance with techniques described in this disclosure.

FIG. 16 is an illustration of an example eye region including patch, in accordance with techniques described in this disclosure.

FIG. 17 is an illustration of an example eye region including patch, in accordance with techniques described in this disclosure.

### DETAILED DESCRIPTION

In some examples, the disclosure describes systems, devices, and techniques for evaluating a patient's depth of anesthesia (DOA), before, during, and/or following a medical procedure (e.g., a surgical procedure during which the patient is operated on by a surgeon). For example, such an evaluation may be performed on the patient pre-operatively and/or during the medical procedure while the patient is anesthetized. The evaluation may be expressed as a DOA index score that reflects the relative DOA for a patient.

The DOA determination may be helpful for avoiding various adverse reactions or situations, such as, but not limited to, intraoperative awareness with recall, prolonged recovery, and/or an increased risk of postoperative complications for a patient, such as post-operative delirium. In some examples, DOA monitoring may improve patient treatment and outcomes by reducing the incidences of intraoperative awareness, minimizing anesthetic drug consumption, and resulting in faster patient wake-up and recovery.

High frequency ocular microtremors (OMTs) may be caused by extra-ocular muscle activity stimulated by impulses emanating in the brain stem. In some examples, the frequency of these tremors may be correlated to a DOA. For example, in patients whose consciousness is reduced by anesthesia or head injury, the frequency of OMTs may be reduced.

Ocular microtremors may be measured using a variety of methods. For example, OMTs may be measured using a piezo-electric sensor attached to a rod resting on the eyeball, or a piezo-electric sensor attached to the eyelid. Such methods require contact with a patient's eye, for example, by a probe or rod, in order to measure and/or monitor OMTs. Monitoring OMTs using contact methods may cause discomfort in patients, risk injury to a patient's eye, and may require extra equipment and complexity in keeping the patient's head still to ensure the accuracy of the measurement and not injure the patient's eye. Further, monitoring the DOA of a patient via contact OMT measurement methods may also require equipment to be in contact with the patient's eye or eyes during the monitoring period, e.g. during a medical procedure. Such equipment (e.g. rod, probe, and/or the like) may be an extra obstacle in completing a procedure, may get in the way of the procedure, and may place extra constraints and/or requirements on the medical procedure itself to not disturb the contact OMT monitoring/measurement system.

The present disclosure is directed to a non-contact method and apparatus for measuring and monitoring ocular microtremors. In an example method, a sequence of images at or near one or both eyes may be captured. The sequence of images may include features at or near the eye, for example, eyelashes, skin wrinkles, blemishes on the eyelid or near the eye, etc. In some examples, one or more features may be added at or near the eye, for example, reflective materials such as glitter, markings such as from a pen, crayon, marker, highlighter, and/or the like, or a light source such as a light emitting diode (LED). In the example method, the sequence of images may include the motion of the feature or features at or near the eye due to ocular microtremors (OMTs). The motion of the feature or features may comprise a signal that may be extracted from the sequence of images. The extracted signal may contain information relating to OMTs, for example baseline microtremor activity and intermittent tremor bursts. In an example method, the signal may be filtered to determine characteristics of the OMTs, for example, the amplitude and frequency of the OMTs. The DOA of a patient may be inferred from OMT characteristics, for example a reduced OMT frequency, and OMT characteristics may be monitored over time.

The examples disclosed herein provide for non-contact OMT monitoring and may reduce or eliminate the risk of discomfort and injury to a patient's eye, the complexities of measurement system alignment so as to maintain contact with a patient's eye for OMT monitoring, and the constraints and/or requirements placed on completing the medical procedure itself related to ensuring the integrity of OMT monitoring. As described herein, a patient's DOA may be determined based on OMT of the patient monitored using the non-contact techniques described herein.

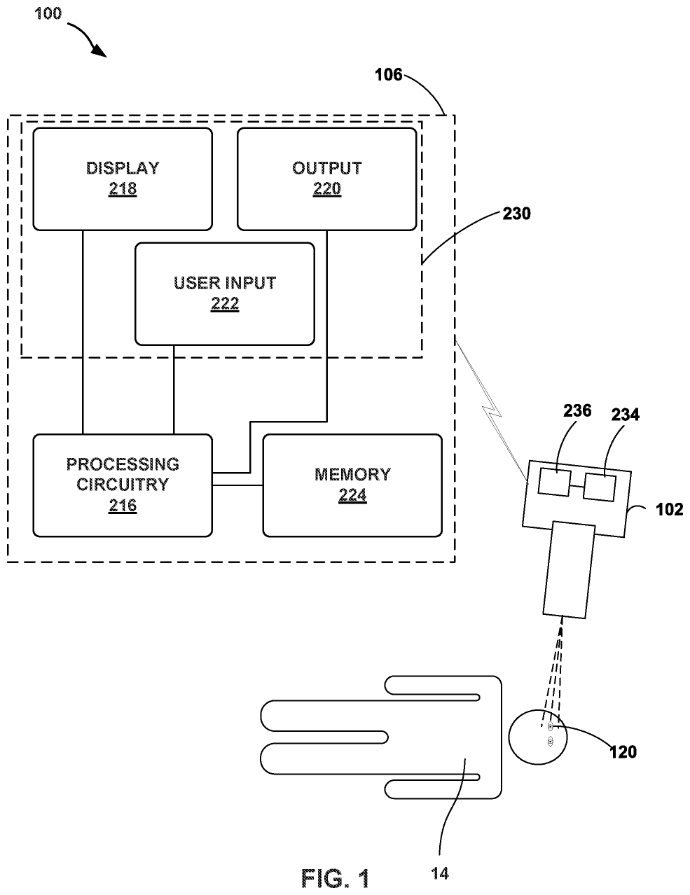

FIG. 1 is an illustration depicting an example ocular microtremor (OMT) monitoring system 100, in accordance with the techniques described in this disclosure. In the example shown, OMT monitoring system 100 includes image captured device 102 and computing device 106. Image capture device 102 may be communicatively coupled, for example by a wired or a wireless connection, to computing device 106. In the illustrated example, computing device 106 may include processing circuitry 216 coupled to display 218, output 220, and user input 222 of a user interface 230.

In some examples, image capture device 102 may be configured to capture a sequence of images of eye region 120 of patient 14. The sequence of images may be transferred to computing device 106 for processing, for example, by a wired or wireless connection between image capture device 102 and computing device 106. In some examples, image captured device 102 may include processing circuitry 236 and memory 234 and may process the sequence of images without transferring the sequence to computing device 106. Image capture device 102 may be any type of camera or video camera capable of capturing a sequence of images. The sequence of images may be two or more images taken at regular or irregular intervals. For example, a sequence of images may include a video stream of images taken at 200 Hz, 350 Hz, 500 Hz, 1000 Hz, or at any other frequency able to resolve motion of features included in the sequence of images related to OMTs.

Processing circuitry 216 of computing device 106, as well as processing circuitry 236 and other processing modules or circuitry described herein, may be any suitable software, firmware, hardware, or combination thereof. Processing circuitry 216 may include any one or more microprocessors, controllers, digital signal processors (DSPs), application specific integrated circuits (ASICs), field-programmable gate arrays (FPGAs), or discrete logic circuitry. The functions attributed to processors described herein, including processing circuitry 216, may be provided by processing circuitry of a hardware device, e.g., as supported by software and/or firmware.

In some examples, processing circuitry 216, as well as processing circuitry 236, is configured to determine physiological information associated with patient 14. For example, processing circuitry 216 may determine an OMT frequency, and/or OMT frequencies, based on a sequence of images of eye region 120, and determine a DOA index score based on the OMT frequency or frequencies, or any other suitable physiological parameter, such as those described herein. Processing circuitry 216 may perform any suitable signal processing of a sequence of images to filter the sequence of images, such as any suitable band-pass filtering, adaptive filtering, closed-loop filtering, any other suitable filtering or processing as described herein, and/or any combination thereof. Processing circuitry 216 may also receive input signals from additional sources (not shown). For example, processing circuitry 216 may receive an input signal containing information about treatments provided to the patient. Additional input signals may be used by processing circuitry 216 in any of the calculations or operations it performs in accordance with OMT monitoring system 100. In some examples, processing circuitry 216 may be adapted to execute software, which may include an operating system and one or more applications, as part of performing the functions described herein. In some examples, processing circuitry 216 may include one or more processing circuitry for performing each or any combination of the functions described herein.

In some examples, processing circuitry 216 may be coupled to memory 224, and processing circuitry 236 may be coupled to memory 234. Memory 224, as well as memory 234, may include any volatile or non-volatile media, such as a random-access memory (RAM), read only memory (ROM), non-volatile RAM (NVRAM), electrically erasable programmable ROM (EEPROM), flash memory, and the like. Memory 224 may be a storage device or other non-transitory medium. Memory 224 may be used by processing circuitry 216 to, for example, store fiducial information or initialization information corresponding to physiological monitoring, such as OMT monitoring. In some examples, processing circuitry 216 may store physiological measurements or previously received data from a sequence of images in memory 224 for later retrieval. In some examples, processing circuitry 216 may store determined values, such as DOA index score, or any other calculated values, in memory 224 for later retrieval.

Processing circuitry 216 may be coupled to user interface 230 including display 218, user input 222, and output 220. In some examples, display 218 may include one or more display devices (e.g., monitor, PDA, mobile phone, tablet computer, any other suitable display device, or any combination thereof). For example, display 218 may be configured to display physiological information and a DOA index score determined by OMT monitoring system 100. In some examples, user input 222 is configured to receive input from a user, e.g., information about patient 14, such as age, weight, height, diagnosis, medications, treatments, and so forth. In some examples, display 218 may exhibit a list of values which may generally apply to patient 14, such as, for example, age ranges or medication families, which the user may select using user input 222.

User input 222 may include components for interaction with a user, such as a keypad and a display, which may be the same as display 218. In some examples, the display may be a cathode ray tube (CRT) display, a liquid crystal display (LCD) or light emitting diode (LED) display and the keypad may take the form of an alphanumeric keypad or a reduced set of keys associated with particular functions. User input 222, additionally or alternatively, include a peripheral pointing device, e.g., a mouse, via which a user may interact with the user interface. In some examples, the displays may include a touch screen display, and a user may interact with user input 222 via the touch screens of the displays. In some examples, the user may also interact with user input 222 remotely via a networked computing device.

In some examples, eye region 120 includes any natural features of patient 14, for example, the eyes, eyelids, eyelashes, eyebrows, skin in the area of the eyes, or any feature that may indicate OMTs, for example, by visibly moving. Eye region 120 may also include features added to eye region 120, for example, glitter, a pattern, a light source, an eyepatch including a pattern or a light source, etc.

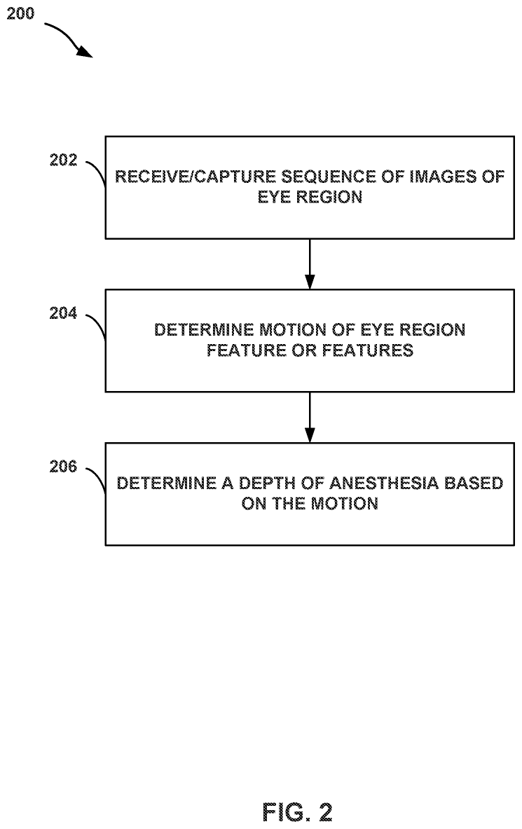

FIG. 2 is a flowchart illustrating an example method 200 of determining a depth of anesthesia, in accordance with techniques described in this disclosure. For purposes of description, the technique of FIG. 2 is described with regard to OMT monitoring system 100. However, the example technique may be employed by any suitable system. The example technique of FIG. 2 may be carried out while patient 14 is anesthetized for a medical procedure.

As shown in FIG. 2, OMT monitoring system 100 may capture a sequence of images of any eye region, for example, via image capture device 102 (202). In some examples, OMT monitoring system 100 may receive a sequence of images of an eye region, for example, at computing device 106 (202). In some examples, the sequence of images may be captured and received continuously and at one or more frame rates. For example, the sequence of images may be continuously captured at a rate of 200 Hz, 350 Hz, 500 Hz, 1000 Hz, or at any other frequency able to resolve motion of features related to OMTs, during a procedure and/or for a period of time before a medical procedure (e.g. several minutes before anesthetization) through a time after a medical procedure as patient 14 comes out of anesthetization. In some examples, the sequence of images may be captured as a “rolling window,” for example, the sequence of images may include a predetermined number of images (or a predetermined time at a particular capture rate) and as a new image is captured, the oldest image in the sequence is dropped, or as a new plurality of images is captured the oldest images equal in number (or time) to the new plurality of images is dropped from the sequence.

After capturing and/or receiving a sequence of images of an eye region, OMT monitoring system 100 may determine motion of one or more features in the eye region based on the sequence of images of the eye region (204). For example, OMT monitoring system 100 may extract one or more features from the sequence of images of the eye region and determine a signal based on the motion of the feature, e.g. the difference in spatial position or orientation of the feature or features between frames of the sequence of images. In some examples, a region of interest within the sequence of images that include one or more features may be determined.

As shown in FIG. 2, processing circuitry 216 may determine a DOA of the patient based on the determined motion (206). In some examples, processing circuitry 216 may determine a DOA index score that is indicative of a relative level of DOA of the patient based on the determined motion. For example, characteristic frequencies associated with OMTs of the one or more features may be determined, such as baseline OMT activity and intermittent tremor bursts. A DOA may be determined based on the characteristic OMT frequencies, and an indicator of the patient's DOA, such as a DOA index score, may be displayed. In some examples, a DOA may be determined based on changes in OMT frequencies.

The generated DOA index score may be a numerical value on a scale used to indicate the relative depth of anesthesia of a patient (e.g., a scale of 1 to 100, wherein an index score of 1 indicates a very low level or substantially no anesthesia of the patient and an index score of 100 indicates a very high level of anesthesia of the patient, or a scale of 1 to 10, or another numerical scale). In some examples, the treatment of the patient before, during, and/or after the medical procedure may be tailored based on the DOA index score. In this manner, the overall treatment of a patient undergoing surgery may be improved by, e.g., by modifying the anesthesia administered to a patient based on a determined DOA index score before, during, and/or after the medical procedure as desired.

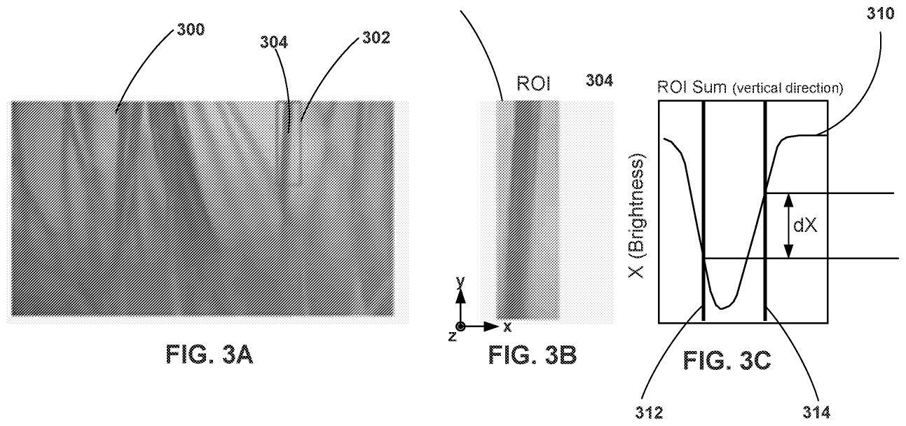

In some examples, a DOA index score may be based on one or more signals relating to OMTs, for example, signals pertaining to, or based on, motion features included in the captured sequence of images as described below with respect to FIGS. 3A-6. For example, a DOA index score may be based on a determined OMT frequency or frequencies such that higher OMT frequencies may indicate a low level or substantially no anesthesia of the patient and may comprise a low DOA index score, whereas lower OMT frequencies may indicate a higher level of anesthesia of the patient and may comprise a high DOA index score. In some examples, the DOA index score may be determined by comparing an initial OMT frequency before the patient is anesthetized, e.g. a DOA index score may be based on comparison of a reference OMT frequency. In some examples, a DOA index score may be determined based on a ratio and/or a difference of OMT frequency, e.g. the current OMT frequency of a patient being monitored by OMT monitoring system 100, to the reference OMT frequency. In some examples, a DOA index score may be determined from a lookup table of OMT frequencies associated with DOA index scores determined from population studies of OMTs versus DOA levels of a plurality of people. In some examples, a threshold OMT frequency may be determined, and a DOA index score may be based on the current OMT frequency of a patient relative to the threshold OMT frequency.

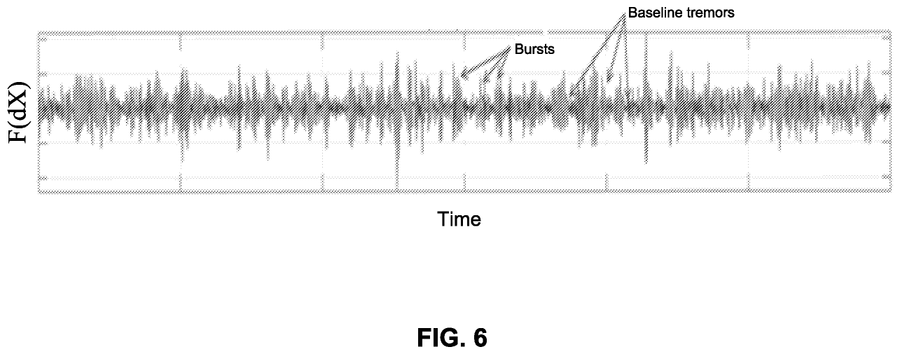

In some examples, a DOA index score may be based on motion features relating to OMTs such as baseline tremors and bursts, as illustrated and described below with respect to FIG. 6. The duration, amplitudes, and frequencies of baseline tremors and bursts may indicate a DOA of a patient, and a DOA index score may be based on the duration, amplitudes, and frequencies of baseline tremors and/or bursts, alone or in combination. In some examples, a DOA index score may be determined by comparing initial OMT baseline tremors and bursts before the patient is anesthetized, e.g. a DOA index score may be based on a comparison of reference baseline tremors and bursts. In some examples, a DOA index score may be determined based on a ratio and/or a difference of current OMT baseline tremors and/or bursts with the reference baseline tremors and/or bursts. In some examples, a DOA index score may be determined from a lookup table of baseline tremors and bursts associated with DOA index scores determined from population studies of OMTs versus DOA levels of a plurality of people. In some examples, threshold baseline tremors and/or bursts may be determined, and a DOA index score may be based on the current baseline tremors and/or bursts of a patient relative to the threshold baseline tremors and/or bursts.

In some examples, a DOA index score may be determined by comparing OMT frequencies and/or baseline tremors and bursts of a patient being monitored to historical data, for example, including the OMT frequencies and/or baseline tremors and bursts of a plurality of patients at a plurality of DOA levels and DOA index scores. In some examples, DOA index score historical data and or lookup tables may further include demographics, e.g. age, weight, sex, body mass index, and the like, of the patient population on which the historical data and/or lookup tables are based, and a DOA index score of a patient currently being monitored may further be based on the patient's demographics.

In some examples, a DOA may be determined based on changes in OMT frequency and/or frequency content. For example, a clinician may observe an indication of OMT frequency and/or frequencies and interpret a DOA based on changes of the OMT frequency and/or frequencies over time.

In some examples, once processing circuitry of a monitoring system, e.g. OMT monitoring system 100, has determined the DOA index score for a patient, the monitoring system may display or otherwise report the determined DOA index score, e.g., to a clinician or other medical personnel. For example, the determined DOA index score may be displayed, e.g. via user interface 230 and display 218. In some examples, the DOA index score may be displayed in terms of the numerical scale, e.g., on a scale of 1 to 100, where 1 indicates the no DOA or the lowest DOA and where 100 indicates the highest DOA for a patient. Alternatively, or additionally, the DOA index score for patient may be indicated via display of a non-numerical technique such as, e.g., using a color scale where different colors correspond to different relative levels of DOA (e.g., green reflecting a desired DOA, and red reflecting an undesirable DOA) or text stating the level of DOA (e.g., “low DOA,” “medium DOA,” or “high DOA”).

In some examples, for patients determined to have a relatively low or high DOA index score, the anesthesia management or protocol in an operating room setting, e.g., type of anesthesia (general, spine) type of drugs used, rate of titration during induction, monitoring of the patient's sedateness, and the like, for the patient may be modified to account for the relatively low or high DOA index score. For example, the DOA monitoring system be configured to provide a recommendation of a course of action to a clinician, e.g., to modify one or more particular parameters (e.g., drug delivery boluses of particular drugs) of anesthesia agents being delivered to the patient to improve the patient's DOA.

FIGS. 3A-5 illustrate an example of a feature captured in an image sequence and signal relating to motion of the feature including OMT frequencies for which a DOA may be determined, for example, by the method 200 performed by a non-contact OMT monitoring system, such as OMT monitoring system 100. More specifically, FIGS. 3A-5 illustrate the processing of a sequence of images that ultimately outputs the temporal frequency spectra of the motion one or more features, the motion including spectral content associated with OMTs that are captured in the image sequence. The processing of the sequence of images illustrated in FIGS. 3A-5 may be performed, for example, by processing circuitry 216 or 236 executing instructions stored in memory 224 or 234.

FIG. 3A is an illustration depicting an example image frame 300 that includes a region of interest (ROI) 302 including a feature 304, in accordance with techniques described in this disclosure. The image frame 300 may be one image of a sequence of images captured, for example, by OMT monitoring system 100, and illustrates a close-up image of eyelashes of a patient, e.g. patient 14, having eyelids closed. Image frame 300 includes feature 304 within ROI 302. The spatial position of feature 304 within the image may change from frame to frame in the image sequence due to motion of the feature. For an image capture device or system that is stationary, the motion of feature 304 captured in the sequence may be caused by motion of the patient, e.g. patient 14 illustrated in FIG. 1. Motion of the patient may include several components, for example, relatively low frequency movements of the patient's head while under anesthesia and relatively higher frequency movements of the patient's eyelid due to OMTs of the patient's eye under the eyelid.

In the example shown, a single eyelash 304 is extracted as illustrated by ROI 302. In some examples, ROI 302 may be predetermined, and features included in ROI 302 may be extracted. In other examples, ROI 302 may be determined by features that are extracted from an image frame.

FIG. 3B is a zoomed-in example of ROI 302 including feature 304, in accordance with techniques described in this disclosure. In some examples, each image frame in the captured or received sequence of images may be stored digitally, e.g. as a spatial distribution of pixels encoding a brightness value at each pixel. The pixel values of the pixels included in ROI 302 illustrated in FIG. 3B are summed to form a sum profile, as illustrated in FIG. 3C.

FIG. 3C is an example plot of a sum profile 310 of ROI 302, in accordance with techniques described in this disclosure. The pixels values of each column of pixels of ROI 302 may be added, e.g. along the y-axis as illustrated in FIG. 3B. The result is a 1D array of summed pixel values associated with the x-axis positions of the columns of ROI 302, e.g. the result is sum profile 310 of ROI 302. FIG. 3C is a plot of sum profile 310. The y-axis of the plot illustrated in FIG. 3C is related to the brightness along each column, and the x-axis is the same as the x-axis of ROI 302 illustrated in FIG. 3B. In the example shown, eyelash 304 is darker than the surrounding area. As such, the sum profile values are lower where the eyelash 304 is located and represents a brightness cross-section of eyelash 304.

In some examples, the motion of eyelash 304 may be determined from sum profile 310. For example, specific positions along the x-axis, positions 312 and 314, may be chosen, e.g. at the nominal left and right edges of eyelash 304 within ROI 302, and the values of sum profile 310 at positions 312, 314 may be determined. The difference between the values at positions 312 and 314 may be determined, e.g. illustrated as sum profile brightness difference dX in FIG. 3C. In the example shown, dX represents the sum profile brightness difference between the vertically summed left and right edges of eyelash 304 at the particular time of the image frame 300 capture. In some examples, the positions 312 and 314 do not correspond to left and right edges of eyelash 304 and may be anywhere along the x-axis of sum profile 310. In some examples, the sum profile brightness difference may be positive or may be negative, e.g. if the sum profile 310 value at position 312 is greater than the value at position 314, dX may be positive and if the sum profile 310 value at position 312 is less than the value at position 314, dX may be negative, or vice versa in some examples. In some examples, dX may be the magnitude, e.g. absolute value, of the sum profile brightness difference and may be only positive.

In the example shown, feature 304 is substantially vertical, that is, the edges of eyelash 304 having a high contrast are substantially vertical, and ROI 302 is a vertical rectangular area of pixels. In other examples, feature 304 may be at any angle relative to the camera pixels, and may be rotated before analysis and summing such that the edges of feature 304 are substantially vertical, e.g. along the y-axis.

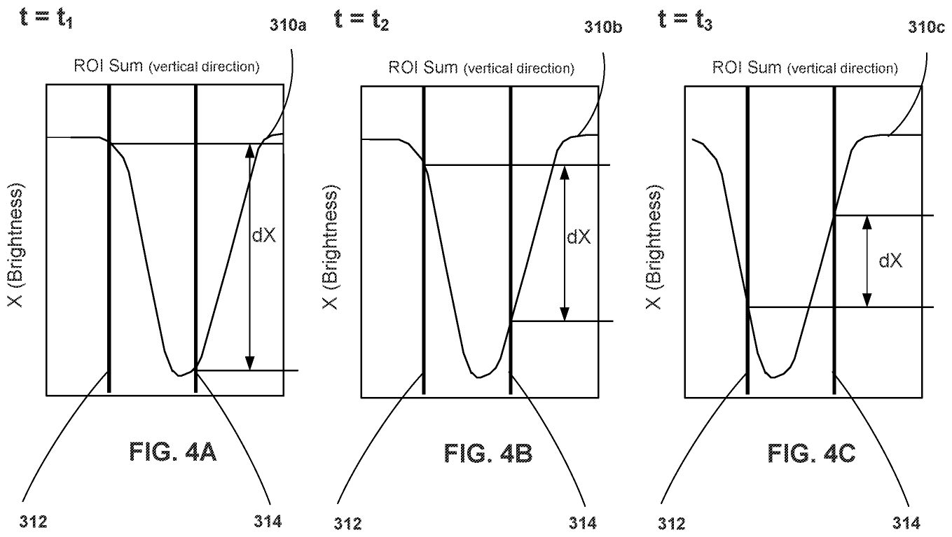

FIGS. 4A-4C are example plots of sum profiles for image frames of the sequence of images captured at three different times, each image frame including feature 304, in accordance with the techniques described in this disclosure. In each of FIGS. 4A-4C, the positions 312 and 314 are the same. FIG. 4A is a plot of sum profile 310a calculated as described above with respect to FIGS. 3A-3C from an image frame captured at time t=t₁, FIG. 4B is a plot of sum profile 310b calculated as described above with respect to FIGS. 3A-3C from an image frame captured at time t=t₂, and FIG. 4C is a plot of sum profile 310c calculated as described above with respect to FIGS. 3A-3C from an image frame captured at time t=t₃, where t₁, t₂, and t₃ are different from each other, e.g. each image frame is a different image frame in the captured or received sequence of images.

In the example shown, the sum profile brightness difference dX changes for each of FIGS. 4A-4C because each sum profile 310a-c shifts along the x-axis, the shifts being due to shifts in the x-axis position of the feature 304, e.g. eyelash 304, in the sequence of images, the shifts of eyelash 304 being due to motion of eyelash 304. As such, the sum profile brightness difference values dX for the sequence of images, e.g. as a function of time, may be a signal pertaining to, or based on, motion of the eyelash 304.

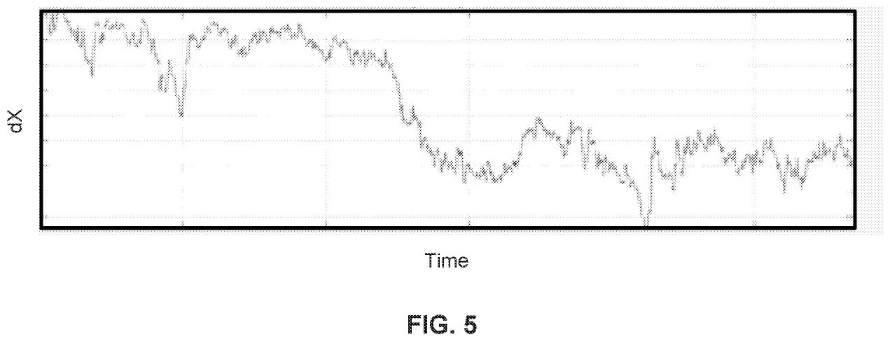

FIG. 5 is an example plot of a signal dX pertaining to motion of a feature included in a sequence of images, in accordance with the techniques described in this disclosure. In the example shown, the values of the signal dX are the values of the sum profile brightness difference dX of each of the image frames of the sequence of images. In the example shown, signal dX includes information relating OMTs, e.g. characteristic frequencies of motion of one or more features ultimately caused by OMTs. In some examples, signal dX may also include information relating to other types of motion that do not relate to OMTs, for example, a shift in the position of the patient's head or face. In some examples, signal dX may be filtered to omit information not relating to OMTs, for example, by high pass filtering or band pass filtering signal dX, as illustrated and described below with respect to FIG. 6.

FIG. 6 is an example plot of a filtered signal F(dX) pertaining to motion of a feature included in a sequence of images, in accordance with the techniques described in this disclosure. In general, any type of filtering may be used to arrive at F(dX), for example high pass or band pass filtering in the Fourier domain and including techniques associated with filtering such as apodization to minimize or eliminate filtering artifacts, filtering utilizing time-frequency analysis for example based upon wavelets, filtering in the time domain, and the like. In general, filtered signal F(dX) includes characteristic frequencies of motion due to OMTs and excludes spectral content of motion due to other sources. In the example shown, the filtered signal F(dX) includes motion features related to OMTs such as baseline tremors and bursts.

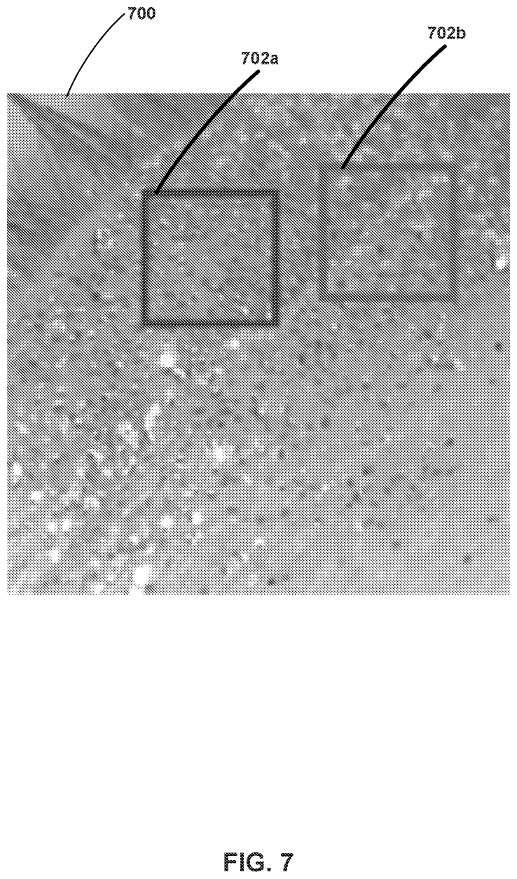

FIG. 7 is an illustration depicting an example image frame that includes a plurality of regions of interest (ROIs) that include features added to the eyelid, in accordance with the techniques described in this disclosure. In the example shown, image frame 700 includes ROI 702a and ROI 702b, each of which are located in different areas of image frame 700. In the example shown, ROIs 702a-b are non-overlapping, are square, and are the same size. In other examples, ROIs 702a-b may be overlapping, may by any shape, and may bound an area that is different from each other.

In the example shown, glitter has been added to the eyelid in image frame 700 as a plurality of features that may be used generate a motion signal, such as the motion signal dX described above with respect to FIGS. 5-6. In some examples, a plurality of motion signals dX for the plurality of features may be derived, for example, by determining a sum profile brightness difference signal as described above, and combined to determine a signal from which characteristic frequencies and features, e.g. the durations, amplitudes, and frequencies of baseline tremors and bursts, of OMTs may be determined. For example, combining a plurality of motion signals for a plurality of features may provide a combined motion signal dX having an increased signal to noise ratio (SNR) from which a DOA index score may be determined. In some examples, the characteristic frequencies and features of OMTs may be determined for each of a plurality of motion signals dX and then combined to determine a combined OMT frequency and/or features.

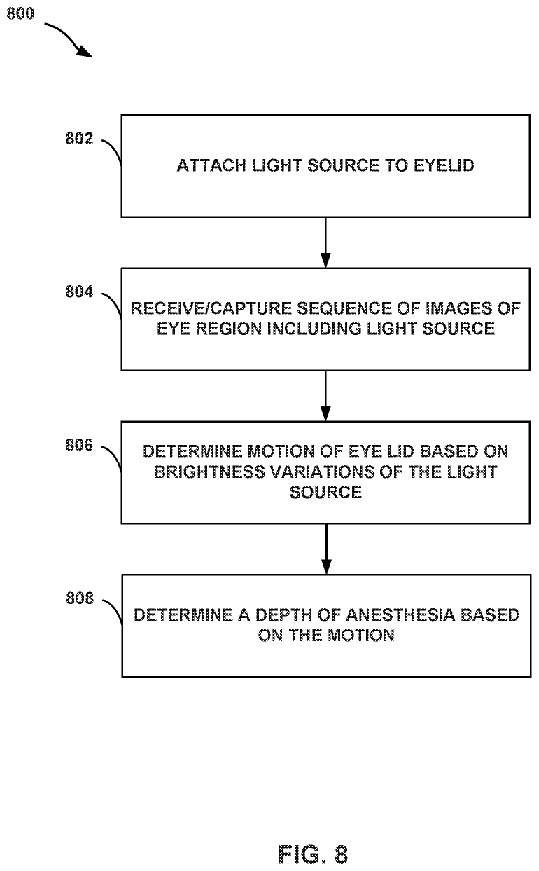

FIG. 8 is a flowchart illustrating an example method 800 of determining a depth of anesthesia of a patient, in accordance with techniques described in this disclosure. For purposes of description, the technique of FIG. 8 is described with regard to OMT monitoring system 100. However, the example technique may be employed by any suitable system. The example technique of FIG. 8 may be carried out while patient 14 is anesthetized for a medical procedure.

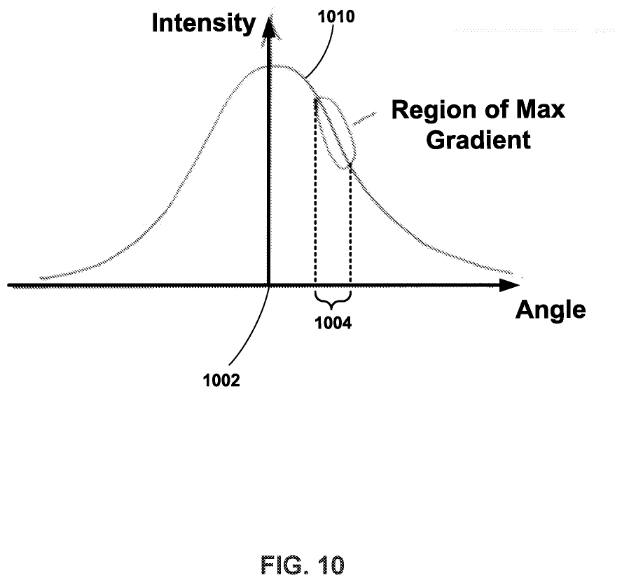

In the example shown, a light source may be attached to an eyelid (802), for example, the eyelid of patient 14 of OMT monitoring system 100 illustrated in FIG. 1. The light source may be any light source, and in some examples the light source is a light emitting diode (LED). In some examples, the light source may have an intensity that varies according to output angle from the direction of maximum intensity output by the light source, as further illustrated and described below with respect to FIGS. 10-13. In some examples, the light source may be attached using a pressure sensitive adhesive, or any other type of appropriate adhesive, disposed on the back of the light source or on an eyelid of the patient. In some examples, the light source may be integrated into a carrier having a pressure sensitive adhesive (or other adhesive), for example, a tape or a patch, that may be attached to an eyelid of a patient. In some examples, the light source may be integrated into a carrier such as an eyepatch that may be secured to a patient's eye, for example, by a strap or any other type of mechanical attachment.

As shown in FIG. 8, OMT monitoring system 100 may capture a sequence of images of the eye region including the light source, for example, via image capture device 102 (804). In some examples, OMT monitoring system 100 may receive a sequence of images of an eye region including the light source, for example, at computing device 106 (804). For example, capturing and/or receiving a sequence of images (804) may be substantially the same as described above with respect to (202) of FIG. 2 where the sequence of images includes the light source which may be a feature that may be used in addition to, or as an alternative to, features described above with respect to FIG. 2.

After capturing and/or receiving a sequence of images of an eye region including the light source, OMT monitoring system 100 may determine motion of one or more features in the eye region based on the sequence of images of the eye region (806). For example, OMT monitoring system 100 may extract one or more features from the sequence of images of the eye region and determine a signal based on the motion of the feature, e.g. the difference in spatial position or orientation of the feature or features between frames of the sequence of images. In some examples, such a feature may be the brightness of the light source attached to the eyelid, and OMT monitoring system 100 may determine brightness variations of the light source within the sequence of images due to variation in orientation of the light source on the eyelid due to motion of the eyelid from OMTs.

Based on the determined motion of one or more features in the eye region, a DOA may be determined based on the determined motion (808). For example, characteristic frequencies associated with OMTs of the one or more features may be determined, such as baseline OMT activity and intermittent tremor bursts. A DOA index score may be determined based on the OMT frequencies, or changes in OMT frequencies over a period of time. For example, determining a DOA index score at (808) may be substantially the same as described above with respect to (206) of FIG. 2 where the sequence of images includes the light source which may be a feature that may be used in addition to, or as an alternative to, features described above with respect to FIG. 2.

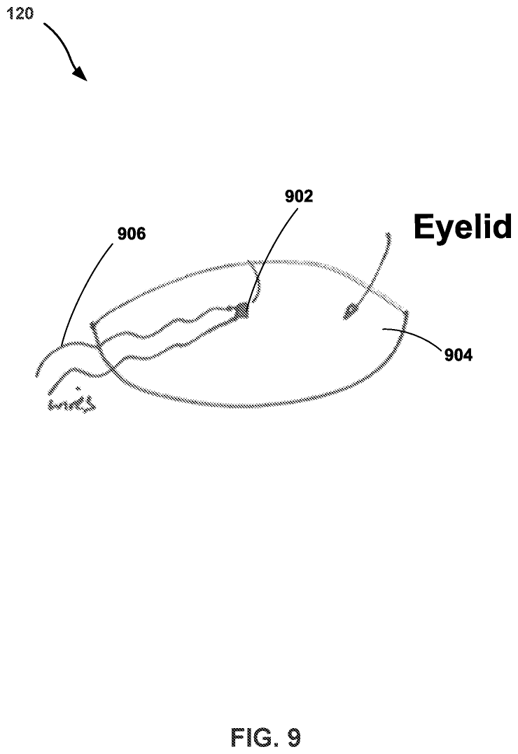

FIGS. 9-13 illustrate example systems and methods for capturing one or more features in an image sequence and determining a signal relating to motion of the feature including OMT frequencies for which a DOA may be determined, for example, by the method 800 performed by a non-contact OMT monitoring system, such as OMT monitoring system 100. More specifically, FIGS. 9-13 illustrate light source output distributions of light sources that may be a feature captured in an image sequence from which a signal or signals relating to motion of the feature including OMT frequencies may be determined, and a system for capturing OMT signals based on light source features. Processing circuitry 216 and/or 236 may analyze the sequence of images including light source features to determine a temporal frequency spectra of the motion of one or more light source features.

FIG. 9 is an illustration of an example eye region 120 including eyelid 904 and light source feature 902, in accordance with techniques described in this disclosure. In the example shown, the light source feature attached to eyelid 904 is a LED 902. LED 902 may be attached to eyelid 904 by any appropriate means, for example, by a removable pressure sensitive adhesive on the back of the LED. In the example shown, LED 902 may be electrically connected to a signal source and/or a power source (not shown), for example, by wires 906. In some examples, LED 902 may be driven by a power source such that the brightness of LED 902 is based on the power applied via wires 906. In some examples, a signal source may vary the power, and therefore the brightness, of LED 902, for example, LED 902 may be strobed and synchronized with image capture of a sequence of images by an image capture device. In some examples, the power and/or driving signal of a power and/or signal source may be determined by processing circuitry 216 or 236.

FIG. 10 is an example plot of a cross-section of the angular intensity profile 1010 of a light source that may be used in a system for capturing OMT signals, in accordance with techniques described in this disclosure. The example shown in FIG. 10 illustrates the intensity of a light source as a function of elevation angle from the direction of maximum intensity output by the light source. For example, the light source may be LED 902 attached to eyelid 904, as illustrated in FIG. 9. The output intensity of LED 902 may be a maximum in an outward perpendicular direction from eyelid 904 at the position of attachment of LED 902 on eyelid 904. As eyelid 904 moves, for example due to OMTs of the patient's eyeball underneath eyelid 904, the orientation of the surface of eyelid 904 changes, and the direction of maximum intensity of LED 902 follows the change in orientation of the surface of eyelid 904.

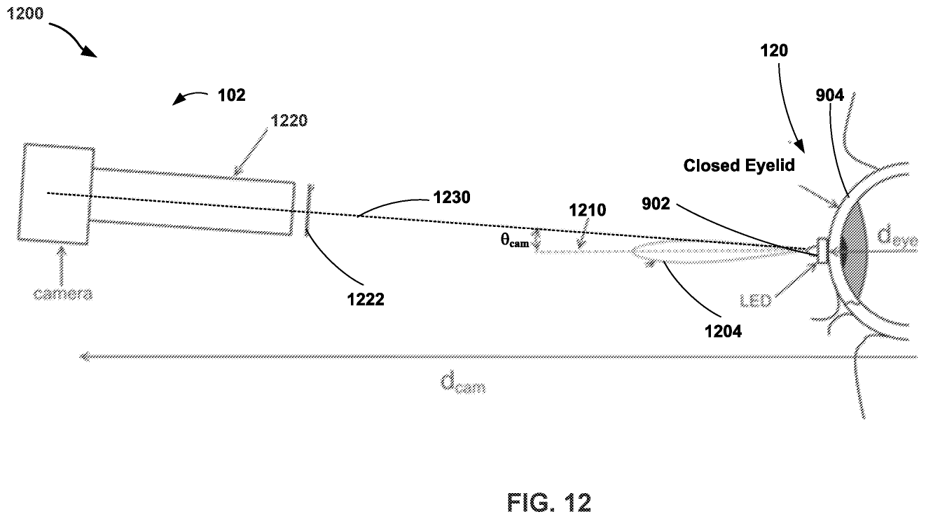

In the example shown, the light source outputs light into a hemisphere outward from the surface of the eyelid to which it is attached. The angular intensity profile 1010 illustrated in FIG. 10 is a cross-section of the intensity output by the light source into that hemisphere. In the examples shown, the intensity is at a maximum at angle 1002. In some examples, the light source is an LED such as LED 902 and angle 1002 is perpendicular from the surface of LED 902. In the example shown, the intensity output profile 1010 is smoothly varying, and has a maximum gradient at angle range 1004, e.g. at range of angles 1004 “off-axis” from angle 1002. In some examples, an image capture device positioned to view the light source at an angle within the range of angles 1004 may have greater sensitivity to changes in light source orientation due to OMTs changing the surface orientation of the eyelid to which the LED is attached, as described in further detail with respect to FIG. 12 below.

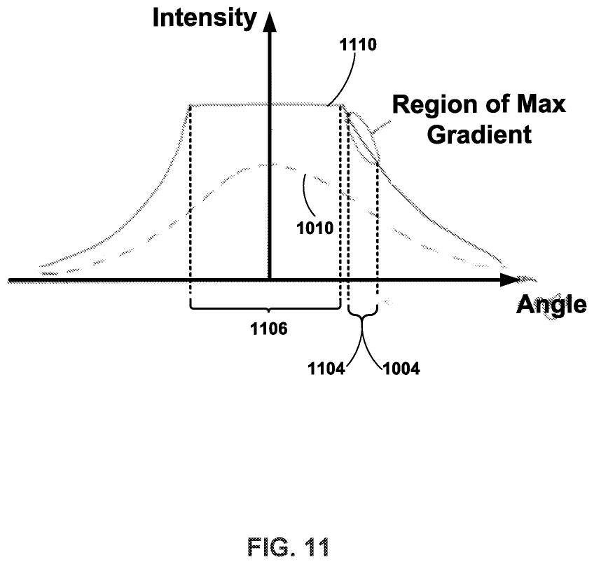

FIG. 11 is an example plot of another cross-section of the angular intensity profile 1110 of a light source that may be used in a system for capturing OMT signals from the perspective of an observing detector, in accordance with techniques described in this disclosure. The example shown in FIG. 11 illustrates the intensity of a light source as a function of elevation angle from the direction of maximum intensity output by the light source, similar to FIG. 10. In the example shown in FIG. 11, the intensity of the light source may be such that its maximum intensity saturates an observing image capture device. As such, for range of angles 1106, the pixels of the observing image capture device will be saturated, e.g. the pixels will output their maximum value for any intensity level above their maximum threshold value.

In the example shown, the image capture device is able to observe the smoothly varying intensity output profile 1110 of the light source at output angles of the light source for which the intensity has dropped off to less than the saturation threshold of the pixels of the image capture device, e.g. angles larger than range of angles 1106. In the example shown, the intensity output profile has a maximum gradient at angle range 1104 and an image capture device positioned to view the light source at an angle within the range of angles 1104. In some examples, by way of comparison between the output intensity profile 1110 and 1010 (repeated as a dashed profile in FIG. 11), the angle range 1004 and 1104 may be the same for each profile. An image capture device positioned to view the light source at an angle within the range of angles 1104 may have greater sensitivity to changes in light source orientation observing the output intensity profile 1110 than the output intensity profile 1010 because the dynamic range of the intensities of the output intensity profile 1110 is greater than that of output intensity profile 1110. For example, at the range of angles 1004, 1104, the slope of output intensity profile 1110 is greater than the slope of output intensity 1010.

FIG. 12 is an illustration of a system 1200 for capturing OMT signals, in accordance with techniques described in this disclosure. In the examples shown, the system 1200 includes image captured device 102, and eye 120.

In the examples shown, eye 120 has a closed eyelid 904 and light source 902 is attached to the outer surface of eyelid 904, for examples, as described above with respect to FIG. 9.

In some examples, light source 902 has a far-field intensity angular output distribution 1204 as illustrated in FIG. 12. Far-field intensity angular output distribution 1204 may have an output intensity profile similar to output intensity profile 1010 or 1110. In other examples, far-field intensity angular output distribution 1204 may have any output intensity profile that varies over angle, e.g. is not collimated. In the examples shown, light source 902 may have an optical axis 1210 that is nominally perpendicular to the surface of eyelid 904 at the position on eyelid 904 at which light source 902 is attached. In the examples shown, far-field intensity angular output distribution 1204 has a maximum along light source optical axis 1210.

In the examples shown, image capture device 102 includes telephoto lens 1220 and optical filter 1222. Image capture device 102 may have an optical axis 1230, and in some examples, image capture device optical axis 1230 may be offset by an angle from light source optical axis 1210, as illustrated in FIG. 12. For example, optical axis 1230 may be configured to observe light source 902 at view angle θ within range of angles 1010 or 1110. In other examples, image capture device 102 may be configured to observe light source 902 at any other angle.

In some examples, the sensitivity of system 1200 to light source intensity changes arising from light source orientation changes due to OMTs may be increased by zooming in on the light source, for example, by using telephoto lens 1220. For example, the use of telephoto lens 1220 allows the distance between image capture device 102 and light source 902 to be increased, which adds leverage to rotational movement and causes the arc length of the light from light source 902 to increase according to a leverage ratio determined by the image capture device 102 distance to the center of the eye 120 and eye 102 radius. Zooming in with telephoto lens 1220 may cause the image of light source 902 to be enlarged, and any movement of light source 902 will be enlarged proportional to the magnification effected by telephoto lens 1220.

In some examples, optical filter 1222 may be included anywhere along the optical path between light source 902 and the imaging elements of image capture device 102, e.g. optical filter may be positioned between telephoto lens 1220 (or any image capture device 102 lens) and the focal plane array of image capture device 102, or optical filter 1222 may be placed within telephoto lens 1220, or optical filter 1222 may be placed between telephoto lens 1220 and light source 902. In some examples, optical filter 1222 is a separate optical element, and in other examples optical filter 1222 may be integrated with other components, such as an optical coating of a window or lens of image capture device 102. In some examples, optical filter 1222 may be a spectral bandpass filter that is spectrally matched to the spectral output of light source 902 and is able to reduce and/or remove noise due to ambient light.

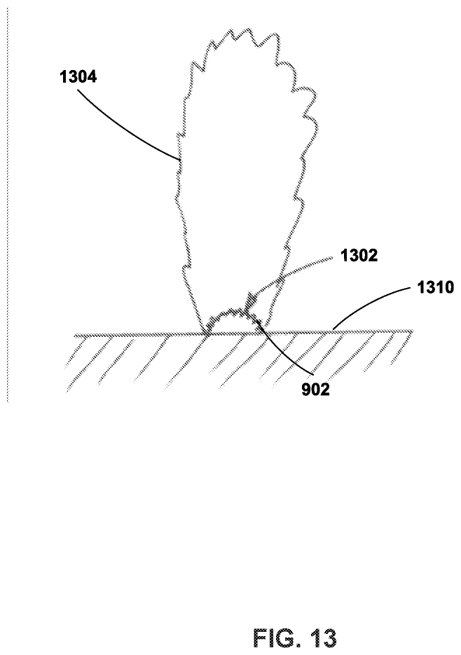

FIG. 13 is a schematic of an example far-field intensity angular output distribution 1304 of a light source 902 that may be used in a system for capturing OMT signals, in accordance with techniques described in this disclosure. The example shown in FIG. 13 includes light source 902 attached to a surface 1310, for example, the surface of eyelid 904.

In the examples shown, the far-field intensity angular output distribution 1304 is irregular. As such, small changes in viewing angle of light source 1302 result in large far-field intensity changes across the range of angles within the hemisphere into which light source 1304 emits light. The sensitivity of an OMT monitoring system, such as OMT monitoring system 100, may be independent of view angle, e.g. θ illustrated in FIG. 12 above. In other words, in contrast to far-field intensity angular output distributions such as 1010 and 1110 having a particular range of observation angles for which sensitivity to brightness change is a maximum for a system capturing OMT signals, far-field intensity angular output distribution 1304 includes a plurality of ranges of observation angles for which sensitivity to brightness change is at or near a maximum, thereby reducing the dependence of sensitivity on the view angle θ of the observing system, for example, system 1200.

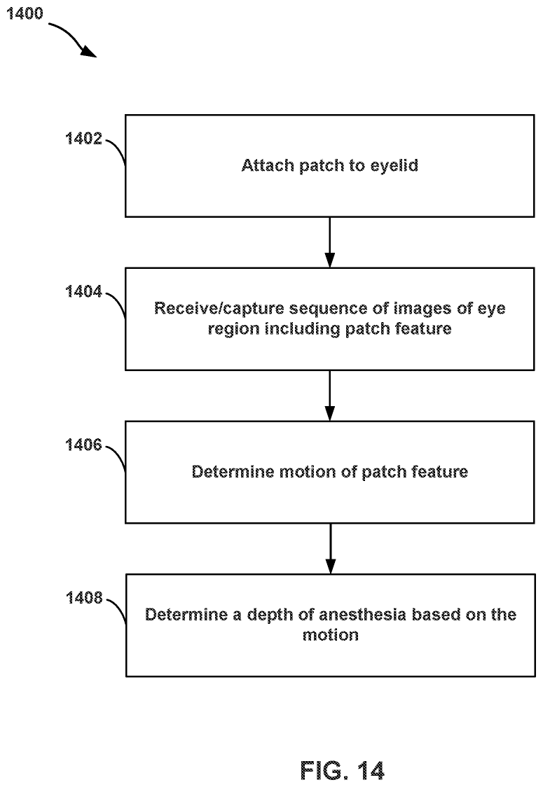

FIG. 14 is a flowchart illustrating an example method 1400 of determining a depth of anesthesia, in accordance with techniques described in this disclosure. For purposes of description, the technique of FIG. 14 is described with regard to OMT monitoring system 100. However, the example technique may be employed by any suitable system. The example technique of FIG. 14 may be carried out while patient 14 is anesthetized for a medical procedure.

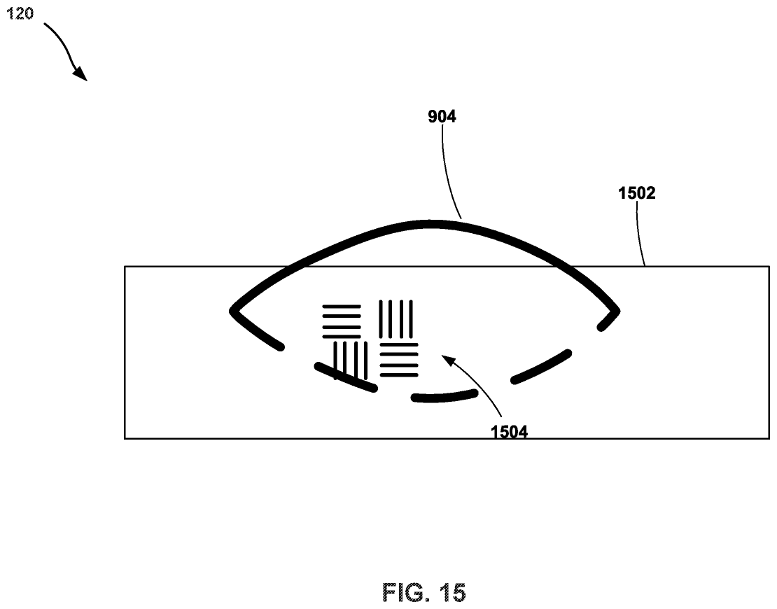

In the example shown, a patch may be attached to an eyelid (1402), for example, the eyelid of patient 14 of OMT monitoring system 100 illustrated in FIG. 1. The patch may include a feature, for example, a light source or a pattern. The light source may be any light source, for example, light source 902, light source 1302, or any other light source. The pattern may be any type of pattern as further illustrated and described below with respect to FIG. 15. In some examples, the patch may be attached using a pressure sensitive adhesive, or any other type of appropriate adhesive, disposed on the back of the patch or on an eyelid of the patient. In some examples, the patch may be secured to a patient's eye, for example, by a strap attached to the patch and fit around the patient's head, or by any other type of mechanical attachment.

As shown in FIG. 14, OMT monitoring system 100 may capture a sequence of images of the eye region including the feature of the patch, for example, via image capture device 102 (1404). In some examples, OMT monitoring system 100 may receive a sequence of images of an eye region including the feature of the patch, for example, at computing device 106 (1404). For example, capturing and/or receiving a sequence of images (1404) may be substantially the same as described above with respect to (202) of FIG. 2 where the sequence of images includes the feature of the patch, which may be a feature that may be used in addition to, or as an alternative to, features described above with respect to FIGS. 2 and 8.

After capturing and/or receiving a sequence of images of an eye region including the feature of the patch, OMT monitoring system 100 may determine motion of one or more features in the eye region based on the sequence of images of the eye region (1406). For example, OMT monitoring system 100 may extract one or more features from the sequence of images of the eye region and determine a signal based on the motion of the feature, e.g. the difference in spatial position or orientation of the feature or features between frames of the sequence of images. In some examples, such a feature may be the brightness of a light source attached to the eyelid via the patch, or brightness variations of a pattern on the patch. OMT monitoring system 100 may determine motion of the eyelid from OMTs based on the brightness variations.

Based on the determined motion of one or more features in the eye region, a DOA may be determined based on the determined motion (1408). For example, characteristic frequencies associated with OMTs of the one or more features may be determined, such as baseline OMT activity and intermittent tremor bursts. In some examples, a DOA index score may be determined based on the OMT frequencies. For example, determining a DOA index score at (1408) may be substantially the same as described above with respect to (206) of FIG. 2 where the sequence of images includes the feature of the patch which may be a feature that may be used in addition to, or as an alternative to, features described above with respect to FIGS. 2 and 8.

FIGS. 15-17 illustrate example patches, including examples features that may be captured in an image sequence for which a signal relating to motion of the feature, including OMT frequencies, for which a DOA may be determined, for example, by the method 1400 performed by a non-contact OMT monitoring system, such as OMT monitoring system 100.

FIG. 15 is illustration of an example eye region 120 including eyelid 904 and patch 1502, in accordance with techniques described in this disclosure. In the example shown, the patch 1502 is attached to eyelid 904 and includes a feature, e.g. pattern 1504. In the example shown, pattern 1504 is a series of vertical and horizontal lines. The lines of pattern 1504 may be used to determine an OMT signal, for example, by using the method 200 described above with respect to FIG. 2. In some examples, each line of pattern 1504 may provide an OMT signal, and through averaging of each signal the resulting averaged OMT signal may be denoised. In some examples, the lines may all be vertical, horizontal, or at any other angle. In some examples, the lines may be orthogonal to each other and at an angle other than vertical and horizontal. In still other examples, there may be any number of series of parallel lines offset from each other by any angle. In some examples, pattern 1504 including series of lines at least at two orthogonal angles with respect to each other allows for orthogonal motion to be captured.

In some examples, patch 1502 may be surgical tape, which may concurrently be used to keep eyelid 904 closed during a procedure.

In some examples, pattern 1504 may be a barcode that allows image capture device 102 to operate and/or includes identifying information, for example, patient identification, billing information, procedure type and details, etc.

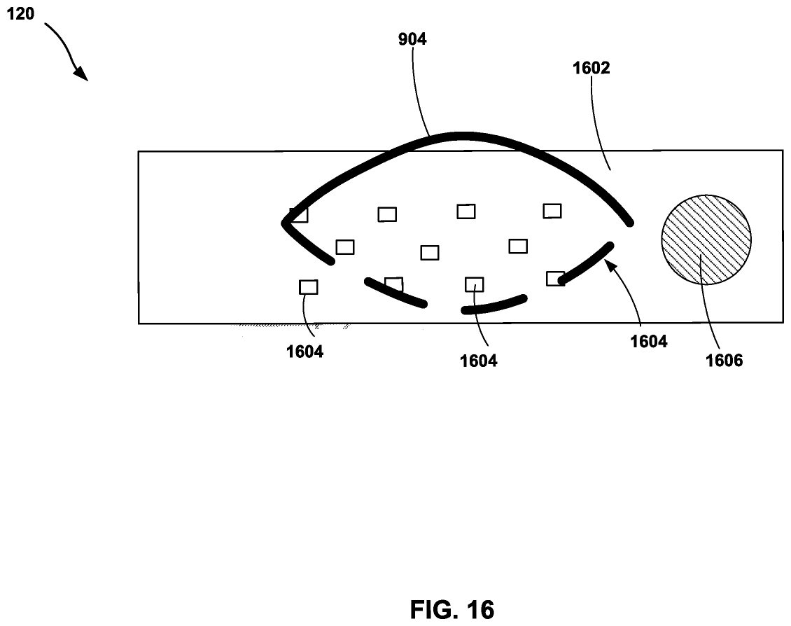

FIG. 16 is an illustration of an example eye region 120 including eyelid 904 and patch 1602, in accordance with techniques described in this disclosure. In the example shown, the patch 1602 is attached to eyelid 904 and includes one or more features, e.g. light source features 1604, and a power supply 1606. In the example shown, the light sources 1604 are attached to patch 1602 and wires delivering electrical power to light sources 1604 may be incorporated within patch 1602 and connected to power supply 1606. Power supply 1606 may be attached to patch 1602, for example, via a housing attached to patch 1602. In some examples, power supply 1606 may be remote to patch 1602, and/or patch 1602 may not include power supply 1606. Power may be provided to patch 1602 and light source features 1604 wirelessly, e.g. via inductive coupling, illumination of a photovoltaic cell, and the like. In the example shown, light sources 1604 may be used to determine an OMT signal, for example, by using the method 800 described above with respect to FIG. 8. In some examples, patch 1602 may be surgical tape, which may concurrently be used to keep eyelid 904 closed during a procedure.

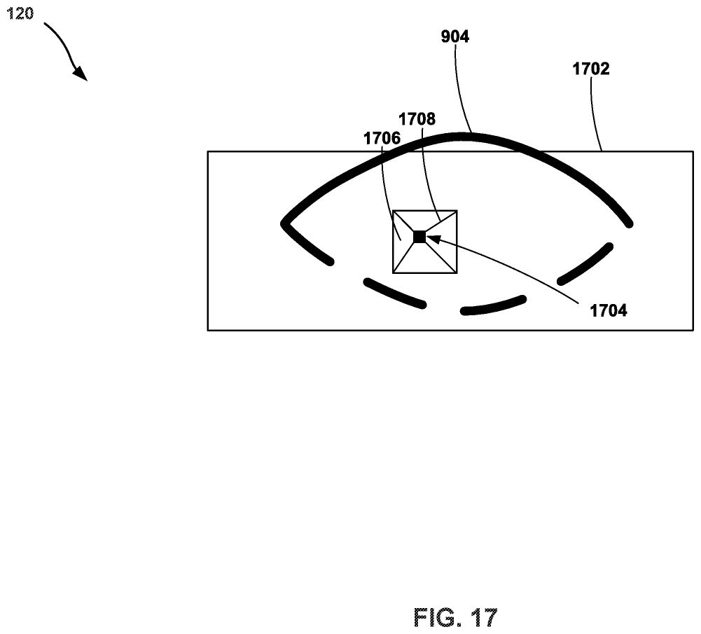

FIG. 17 is an illustration of an example eye region 120 including eyelid 904 and patch 1702, in accordance with techniques described in this disclosure. In the example shown, the patch 1702 is attached to eyelid 904 and includes one or more features, e.g. light source feature 1704. In the example shown, the light source 1704 may be incorporated within patch 1702 in the area of a transparent window 1706 and suspended within the area of transparent window 1706, for example, by wires 1708. In some examples, transparent window 1706 may be a transparent material, and in other examples, transparent window 1706 may be an open area, e.g. a region in which the material of patch 1702 has been removed. In the example shown, light source 1704 is configured to move with the eyelid to which patch 1702 is attached without being rigidly attached to patch 1702 so as to avoid any damping or reduction in transfer of motion of the underlying eyelid to light source 1704 by patch 1702. For example, the back of light source 1704 may be in contact with the underlying eyelid. In some examples, patch 1702 may include a plurality of transparent areas 1706 including suspended light sources 1704. In the example shown, light sources 1704 may be used to determine an OMT signal, for example, by using the method 800 described above with respect to FIG. 8. In some examples, patch 1702 may be surgical tape, which may concurrently be used to keep eyelid 904 closed during a procedure.

The techniques described in this disclosure may be implemented, at least in part, in hardware, software, firmware or any combination thereof. For example, various aspects of the described techniques may be implemented within one or more processors, including one or more microprocessors, DSPs, application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

The following examples illustrate example subject matter described herein.

Example 1: A method comprising: receiving, from an image capture device, a sequence of images of an eye region of a patient; determining, using processing circuitry, a motion of a feature within the eye region based on the received sequence of images; and determining, using the processing circuitry, a depth of anesthesia of the patient based on the determined motion.

Example 2: The method of example 1, further coprising: extracting at least one feature in a sequence of images of the eye region; determining a signal based on the motion of the feature in the sequence of images; and filtering the signal, wherein determining a depth of anesthesia is based on the filtered signal.

Example 3: The method of example 2, wherein determining the signal based on the motion of the at least one feature in the sequence of images further comprises: determining a region of interest including the at least one feature; summing the pixels of the region of interest along a direction in each of the images of the sequence of images to obtain a one-dimensional sum signal; determining a difference between the values of the summed pixels at two points along the one-dimensional sum signal for each of the images of the sequence of images; and determining the signal based on the determined differences for each of the images of the sequence of images.

Example 4: The method of any of examples 1 through 3, further comprising tracking the feature to keep the feature within the field of view of an image capture system, wherein the sequence of images is captured by the image capture system.

Example 5: The method of any of examples 1 through 4, wherein the at least one feature comprises a high contrast object added to the eye region.

Example 6: The method of any of examples 1 through 5, wherein the at least one feature is located in a plurality of regions of interest within each image of the sequence of images.

Example 7: The method of any of examples 1 through 6, further comprising a composite signal based on a combination of determined signals from a plurality of regions of interest.

Example 8: The method of any of examples 1 through 7, further comprising: attaching a light source to an eyelid in the eye region, the light source configured to emit light in a range of angles outward from the eyelid; positioning the image capture device to capture a sequence of images of the eyelid and the light from the light source; determining a signal based on brightness variations of the light source captured in the sequence of images; filtering the signal; and determining a depth of anesthesia based on the filtered signal.

Example 9: The method of example 8, wherein the image capture device is configured to view the light source at a view-angle that is offset from the angle of far-field intensity maximum of the light source.

Example 10: The method of example 9, wherein the view-angle corresponds to the angle at which the gradient of the light source far-field intensity is at a maximum.

Example 11: The method of example 8 or example 9, wherein the surface of the light source is striated and the far-field intensity of the light source as a function of angle is irregular due to the striations.

Example 12: The method of any of examples 8 through 11, wherein the image capture device includes a telephoto lens.

Example 13: The method of any of examples 8 through 12, wherein the image capture device includes a spectral bandpass filter corresponding to the spectral output of the light source.

Example 14: The method of any of examples 1 through 13, further comprising determining a score indicating the depth of anesthesia based on the signal.

Example 15: A system comprising: an image capture device; and processing circuitry configured to: receive, from an image capture device, a sequence of images of an eye region from the image capture device; determine a motion of a feature within the eye region based on the received sequence of images; and determine a depth of anesthesia based on the determined motion.

Example 16: The system of example 15, wherein the processing circuitry is further configured to: extract at least one feature in a sequence of images of the eye region; determine a signal based on the motion of the feature in the sequence of images; and filter the signal, wherein the determination of a depth of anesthesia is based on the filtered signal.

Example 17: The system of example 16, wherein the processing circuitry is further configured to: determine a region of interest including the at least one feature; sum the pixels of the region of interest along a direction in each of the images of the sequence of images to obtain a one-dimensional sum signal; determine a difference between the values of the summed pixels at two points along the one-dimensional sum signal for each of the images of the sequence of images; and determine the signal based on the determined differences for each of the images of the sequence of images.

Example 18: The system of example 17, further comprising: an ocular microtremor probe comprising a pattern disposed in an eye region, the pattern including at least one feature captured in the sequence of images, wherein an ocular microtremor signal is determined based on the at least one feature.

Example 19: The system of example 18, wherein the pattern disposed in the eye region is disposed on a patch configured to be attached to a person's eyelid.

Example 20: The system of example 18 or example 19, wherein the pattern disposed in the eye region includes a barcode including identification information of the patient and/or including trigger information for a depth of anesthesia system.

Example 21: The system of any of examples 18 through 20, wherein the pattern disposed in the eye region is comprised of a plurality of parallel line sets, the line sets rotated relative to each other at an angle.

Example 22: The system of any of examples 18 through 21, wherein the pattern disposed in the eye region is comprised of at least one light source.

Example 23: The system of example 22, wherein the at least one light source is a light emitting diode (LED), wherein the ocular microtremor probe further comprises a power source electrically coupled to the at least one LED.

Example 24: The system of example 23, wherein the LED is placed within a transparent area in the patch and in contact with the person's eyelid.

Example 25: A method comprising: receiving a sequence of images of an eye region; and determining a motion of a feature within the eye region based on the received sequence of images.

Example 26: The method of example 25, further comprising: extracting at least one feature in a sequence of images of the eye region; determining a signal based on the motion of the feature in the sequence of images; and filtering the signal.

Example 27: The method of example 26, wherein determining the signal based on the motion of the at least one feature in the sequence of images further comprises: determining a region of interest including the at least one feature; summing the pixels of the region of interest along a direction in each of the images of the sequence of images to obtain a one-dimensional sum signal; determining a difference between the values of the summed pixels at two points along the one-dimensional sum signal for each of the images of the sequence of images; and determining the signal based on the determined differences for each of the images of the sequence of images.

Example 28: The method of any of examples 25 through 27, further comprising determining, using the processing circuitry, a depth of anesthesia of the patient based on the determined motion.

Example 29: The method of example 28, wherein determining a depth of anesthesia is based on the filtered signal.

Example 30: A system comprising: an image capture device; and processing circuitry configured to: receive, from an image capture device, a sequence of images of an eye region from the image capture device; and determine a motion of a feature within the eye region based on the received sequence of images.

Example 31: The system of example 30, wherein the processing circuitry is further configured to: extract at least one feature in a sequence of images of the eye region; determine a signal based on the motion of the feature in the sequence of images; and filter the signal.

Example 32: The system of example 31, wherein the processing circuitry is further configured to: determine a region of interest including the at least one feature; sum the pixels of the region of interest along a direction in each of the images of the sequence of images to obtain a one-dimensional sum signal; determine a difference between the values of the summed pixels at two points along the one-dimensional sum signal for each of the images of the sequence of images; and determine the signal based on the determined differences for each of the images of the sequence of images.

Example 33: The system of any of examples 30 through 32, wherein the processing circuitry is further configured to: determine a depth of anesthesia of the patient based on the determined motion.

Example 34: The system of example 33, wherein determining a depth of anesthesia is based on the filtered signal.

Example 35: A system configured to perform one or more of the example techniques described in the disclosure.

Various examples have been described. These and other examples are within the scope of the following claims.

1. A system comprising:
an image capture device;
a light emitting diode (LED) configured to be disposed in an eye region, wherein the image capture device is configured to view the LED at a view-angle that is offset from an angle of far-field intensity maximum of the LED; and
processing circuitry configured to:
receive, from an image capture device, a sequence of images of the eye region from the image capture device;
determine a motion of a feature within the eye region based on the received sequence of images; and
determine a depth of anesthesia based on the determined motion.

2. The system of claim 1, wherein the processing circuitry is further configured to:
extract at least one feature in a sequence of images of the eye region;
determine a signal based on the motion of the feature in the sequence of images; and
filter the signal,
wherein the determination of a depth of anesthesia is based on the filtered signal.

3. The system of claim 2, wherein the processing circuitry is further configured to:
determine a region of interest including the at least one feature;
sum the pixels of the region of interest along a direction in each of the images of the sequence of images to obtain a one-dimensional sum signal;
determine a difference between the values of the summed pixels at two points along the one-dimensional sum signal for each of the images of the sequence of images; and
determine the signal based on the determined differences for each of the images of the sequence of images.

4. The system of claim 3, further comprising:
an ocular microtremor probe comprising a pattern disposed in an eye region, the pattern including at least one feature captured in the sequence of images, wherein an ocular microtremor signal is determined based on the at least one feature.

5. The system of claim 4, wherein the pattern disposed in the eye region is disposed on a patch configured to be attached to a person's eyelid.

6. The system of claim 4, wherein the pattern disposed in the eye region includes a barcode including identification information of the patient and/or including trigger information for a depth of anesthesia system.

7. The system of claim 4, wherein the pattern disposed in the eye region is comprised of a plurality of parallel line sets, the line sets rotated relative to each other at an angle.

8. The system of claim 4, wherein the pattern disposed in the eye region is comprised of at least one light source.

9. The system of claim 8, wherein the at least one light source is the LED, wherein the ocular microtremor probe further comprises a power source electrically coupled to the at least one LED.

10. The system of claim 9, wherein the LED is placed within a transparent area in the patch and in contact with the person's eyelid.

11. The system of claim 1, wherein the processing circuitry is further configured to track the at least one feature and to cause the image capture device to keep the at least one feature within the field of view of an image capture device.

12. The system of claim 1, wherein the at least one feature comprises a high contrast object added to the eye region.

13. The system of claim 1, wherein the at least one feature is located in a plurality of regions of interest within each image of the sequence of images.

14. The system of claim 1, further comprising a composite signal based on a combination of determined signals from a plurality of regions of interest.

15. (canceled)

16. A method comprising:
attaching a light source to an eyelid in an eye region of a patient, the light source configured to emit light in a range of angles outward from the eyelid;
positioning an image capture device to capture a sequence of images of the eyelid and the light from the light source;
receiving, from an image capture device, a sequence of images of the eye region of the patient;
determining, using processing circuitry, a signal based on brightness variations of the light source captured in the sequence of images;
filtering, using the processing circuitry, the signal; and
determining a depth of anesthesia of the patient based on the filtered signal.

17. The method of claim 16, wherein the image capture device is configured to view the light source at a view-angle that is offset from an angle of a far-field intensity maximum of the light source.

18. The method of claim 17, wherein the view-angle corresponds to an angle at which the gradient of the light source far-field intensity is at a maximum.

19. The method of claim 16, wherein a surface of the light source is striated and a far-field intensity of the light source, as a function of angle, is irregular due to the striations.

20. The method of claim 16, wherein the image capture device includes a telephoto lens.

21. The method of claim 16, wherein the image capture device includes a spectral bandpass filter corresponding to a spectral output of the light source.
